(19) United States
(12) Patent Application Publication
(10) Pub. No.: US 2015/0216712 A1
(43) Pub. Date: Aug. 6, 2015

(54) Neck-Worn Arm Rest Device

(71) Applicant: James K. Wilson, JR., Montgomery, AL (US)

(72) Inventor: James K. Wilson, JR., Montgomery, AL (US)

(21) Appl. No.: 14/173,493

(22) Filed: Feb. 5, 2014

(51) Int. Cl. A61F 5/37

(52) U.S. Cl. CPC A61F 5/3738

(57) ABSTRACT

A neck-worn arm rest device receives and supports both of a user's arms proximate the user's chest. The device includes a main panel having a first end and a second end. The main panel is positioned to form a sleeve. The sleeve defines a channel configured for receiving both arms of a user therein to support the arms within the channel. A strap is coupled to and extends outwardly from the main panel. The strap is configured for being worn around a neck of the user. A pocket is coupled to an exterior surface of the main panel. The pocket has an open top end a closed bottom end.

### BACKGROUND OF THE DISCLOSURE

### Field of the Disclosure

The disclosure relates to arm rest devices and more particularly pertains to a new arm rest device for receiving and supporting both of a user's arms proximate the user's chest.

### SUMMARY OF THE DISCLOSURE

An embodiment of the disclosure meets the needs presented above by generally comprising a main panel having a first end and a second end. The main panel is positioned to form a sleeve. The sleeve defines a channel configured for receiving both arms of a user therein to support the arms within the channel. A strap is coupled to and extends outwardly from the main panel. The strap is configured for being worn around a neck of the user. A pocket is coupled to an exterior surface of the main panel. The pocket has an open top end a closed bottom end.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a top front side perspective view of a neck-worn arm rest device according to an embodiment of the disclosure.

FIG. 2 is a rear view of an embodiment of the disclosure.

FIG. 3 is a front view of an embodiment of the disclosure.

FIG. 4 is a side view of an embodiment of the disclosure.

FIG. 5 is an in-use front view of an embodiment of the disclosure.

FIG. 6 is a top front side perspective view of an embodiment of the disclosure showing the pillow positionable within the sleeve.

FIG. 7 is a top front side perspective view of an alternative embodiment of the disclosure.

FIG. 8 is a rear view of an alternative embodiment of the disclosure.

### DESCRIPTION OF THE PREFERRED EMBODIMENT

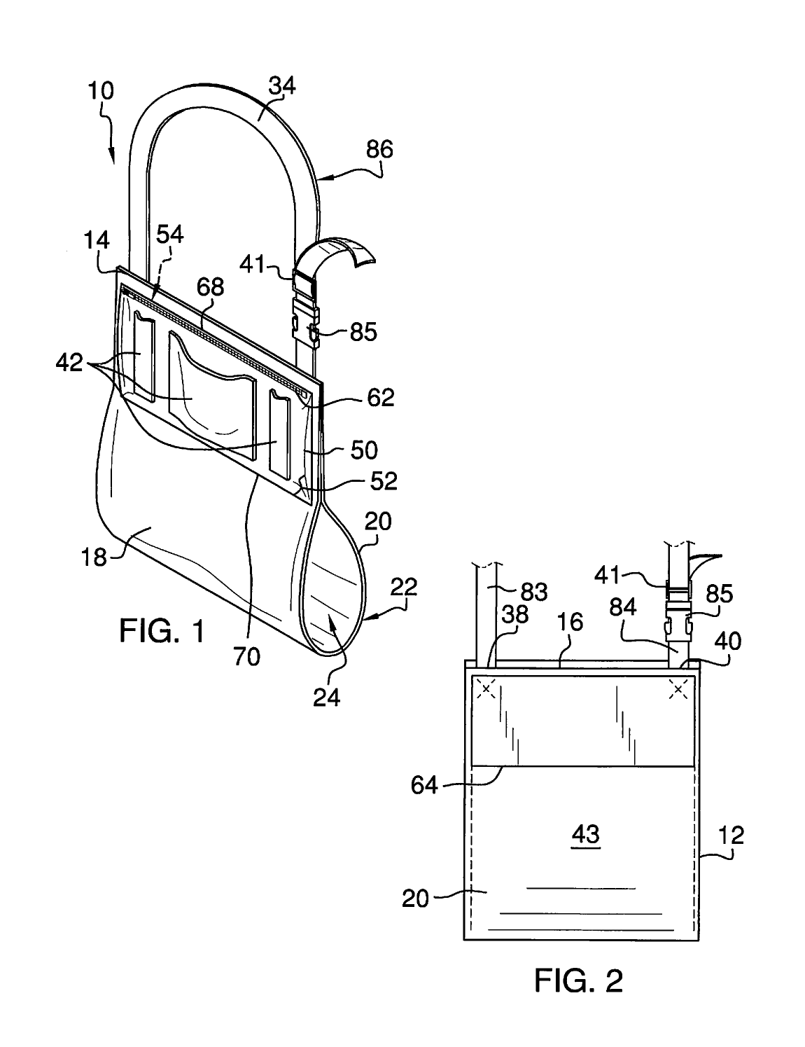

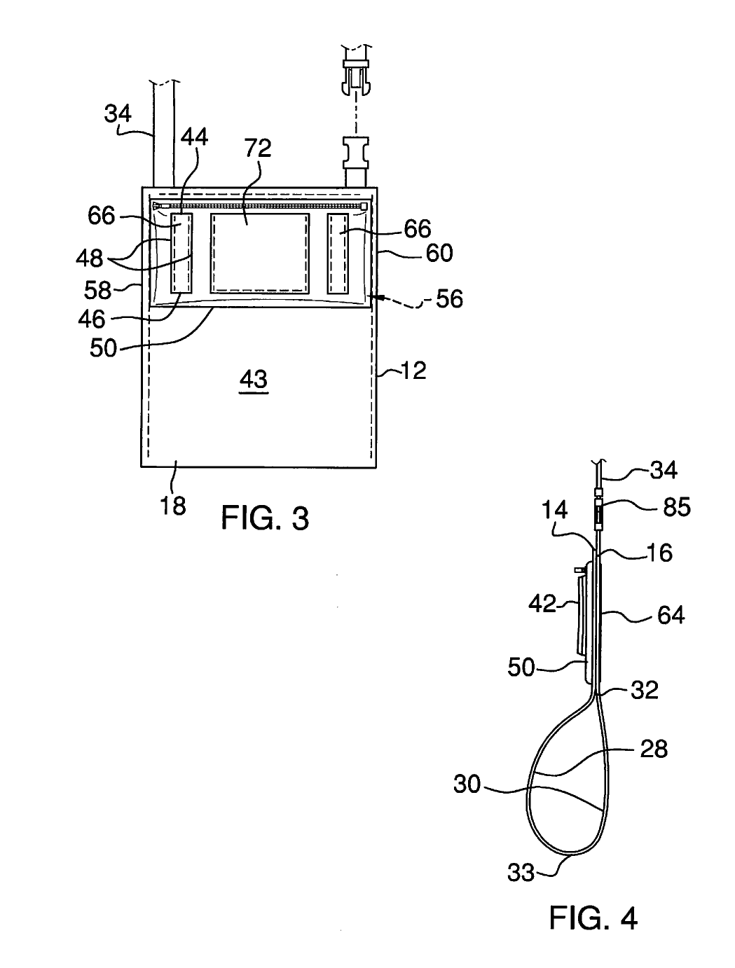

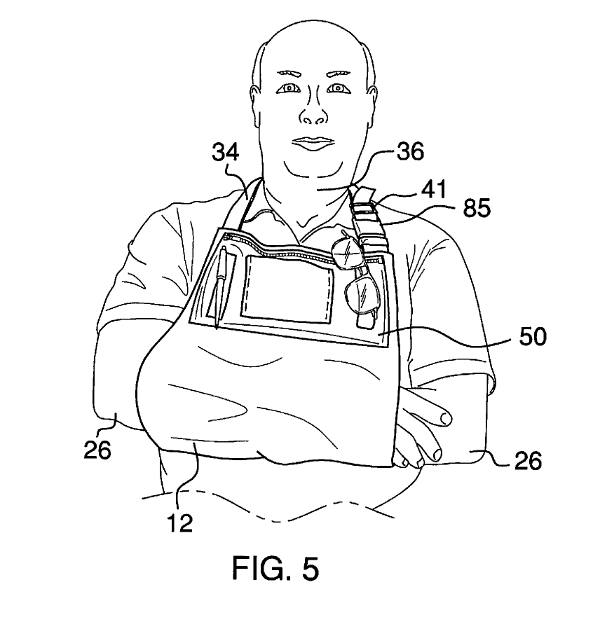

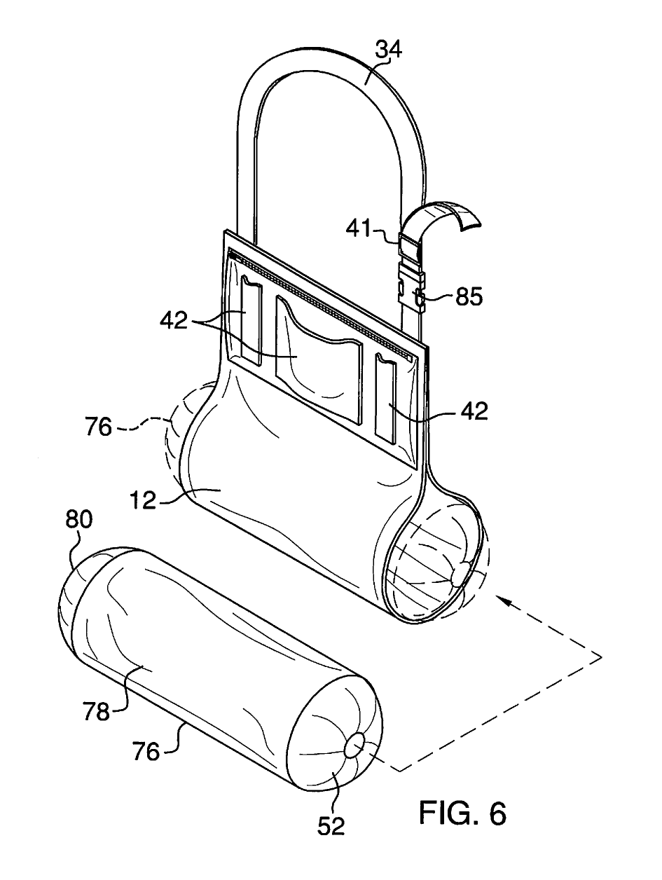

With reference now to the drawings, and in particular to FIGS. 1 through 8 thereof, a new arm rest device embodying the principles and concepts of an embodiment of the disclosure and generally designated by the reference numeral 10 will be described.

As best illustrated in FIGS. 1 through 8, the neck-worn arm rest device 10 generally comprises a main panel 12 having a first end 14 and a second end 16. The main panel 12 is flexible and may be formed from cloth or similar material. The main panel is positioned to form a sleeve 22. The sleeve 22 defines a channel 24 configured for receiving both arms 26 of a user therein to support the arms 26 within the channel 24. The sleeve 22 defines a first section 18 that includes the first end 14 of the main panel 12 and a second section 20 that includes the second end 16 of the main panel 12. The first section 18 may be attached to the second section 20 proximate the first end 14 and the second end 16 of the main panel 12. In particular, an interior surface 28 of the first section 18 may abut an interior surface 30 of the second section 20 between a top 32 of an opened-portion of the channel 24 and each of the first end 14 and the second end 16 of the main panel 12.

A strap 34 is coupled and extends outwardly from the main panel 12. The strap 34 is configured for being worn around a neck 36 of the user. The strap 34 has a first end 38 and a second end 40. Each of the first end 38 and the second end 40 of the strap 34 is attached to the second section 20 of the main panel 12. An adjustment member 41 may be coupled to the strap 34 for selectively adjusting a length of the strap 34. The strap 34 has a break therein defining a first strap portion 83 including the first end 38 of the strap 34 and a second strap portion 84 including the second end 40 of the strap 34. A coupler 85 removably couples the first strap portion 83 to the second strap portion 84 for selectively retaining the strap 34 in a closed loop orientation 86 around the neck 36 of the user. The coupler 85 may comprise a buckle or other conventional fastener.

A pocket 42 is coupled to an exterior surface 43 of the main panel 12. The pocket 42 has an open top end 44 a closed bottom end 46. The pocket 42 may be one of a plurality of pockets 42. Each of the pockets 42 has a pair of sides 48 coupled to and extending between the open top end 44 and the closed bottom end 46 of the associated pocket 42. A pocket panel 50 is coupled to the exterior surface 43 of the main panel 12. Each of the plurality of pockets 42 is attached to an outer surface 52 of the pocket panel 50. The pocket panel 50 has an opening 54 therein for accessing an interior 56 of the pocket panel 50. The pocket panel 50 is positioned on the first section 18 of the main panel 12 proximate the first end 14 of the main panel 12. The pocket panel 50 extends between a first longitudinal side 58 and a second longitudinal side 60 of the main panel 12. A closure 62 is attached to the pocket panel 50 and is actuated to a closed state closing the opening or to an opened state allowing access to the interior 56 of the pocket panel 50. The closure 62 may comprise a zipper, snaps or the like. The closure 62 is positioned above the open top end 46 of the pockets 42 and extends along a length of the pocket panel 50.

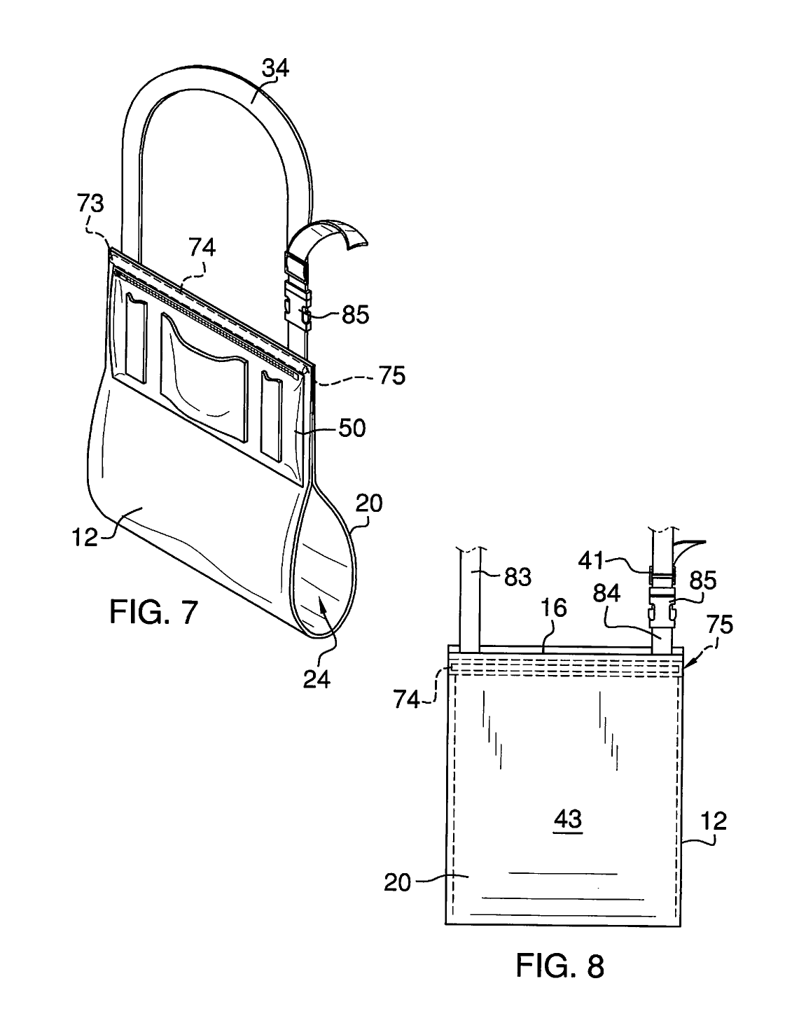

A reinforcement panel 64 is coupled to the exterior surface 43 of the second section 20 of the main panel 12 and is positioned opposite the pocket panel 50. The reinforcement panel 64 is of increased strength relative to the pocket panel 50 and the main panel 12. The sides 48 of a pair 66 of the pockets 42 are elongated and extend vertically between a top 68 and a bottom 70 of the pocket panel 50. In this manner, the pair 66 of the pockets 42 are well-suited for holding elongated items, such as pens, pencils or the like. A third one 72 of the pockets 42 is positioned between the pair 66 of the pockets 42. The sides 48 of the third one 72 of the pockets 42 also extend vertically between the top 68 and the bottom 70 of the pocket panel 50. Alternatively, as shown in FIGS. 7 and 8, a rod 74 may be positioned between the first section 18 and the second section 20 of the main panel 12 in a void 75 extending above the channel 24. Opposite ends of the void 75 may be sewn together to close the void 75 so that the rod 74 is retained within the void 75. The rod 74 abuts the interior surfaces 28, 30 of each of the first section 18 and the second section 20 of the main panel 12. The rod 74 may be constructed from plastic or like material.

A pillow 76 is postionable within the channel 24. The pillow 76 is elongated and has a main section 78, a first end 80 and a second end 82. The main section 78 may be annular. Each of the first end 80 and the second end 82 of the pillow 76 may be convexly arcuate. The first end 80 of the pillow 76 extends outwardly beyond the first longitudinal side 58 of the main panel 12 when the pillow 76 is centrally positioned within the channel 24. Similarly, the second end 82 of the pillow 76 extends outwardly beyond the second longitudinal side 60 of the main panel 12 when the pillow 76 is centrally positioned within the channel 24.

The device 10 may have a length from the first end 14 to the second end 16 of the main panel 12 between approximately 40.0 cm and 45.0 cm. The channel 24 may have an outer diameter from the top 32 of an opened-portion of the channel 24 to a bottom 33 of the channel 24 between approximately 24.0 cm and 35.0 cm. The main section 78 of the pillow 76 may have an outer diameter between approximately 12.0 cm and 18.0 cm. A distance between opposite ends 73 of the rod 74 may measure between approximately 25.0 cm and 38.0 cm.

In use, as stated above and shown in the Figures, the strap 34 is positioned around the neck 36 of the user so that the main panel 12 hangs in front of the user's chest region. The user places desired items, such as travel documents, pens and the like, into the pockets 42 and into the interior 56 of the pocket panel 50. The user can then place both arms 26 into opposite ends of the channel 24 and can fold his or her arms 26 across each other if desired. In this manner, the device 10 provides a surface for resting the user's arms 26, especially while in a crowded setting, such as while seated on an airplane or at a movie theater or even while standing. Alternatively, the user can position the pillow 76 within the channel 24 and place the pillow 76 behind the user's head to provide a comfortable resting surface for the user's head.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A neck-worn arm rest device comprising:
a main panel having a first end and a second end, said main panel being positioned to form a sleeve, said sleeve defining a channel configured for receiving both arms of a user therein to support the arms within said channel;
a strap coupled to and extending outwardly from said main panel, said strap being configured for being worn around a neck of the user; and
a pocket being coupled to an exterior surface of said main panel, said pocket having an open top end a closed bottom end.

2. The device of claim 1, further comprising a pocket panel being coupled to said exterior surface of said main panel, said pocket being attached to an outer surface of said pocket panel.

3. The device of claim 2, further comprising:
said pocket panel having an opening therein for accessing an interior of said pocket panel; and
a closure attached to said pocket panel and being actuated to a closed state closing said opening or to an opened state allowing access to said interior of said pocket panel.

4. The device of claim 3, further comprising wherein said closure comprises a zipper.

5. The device of claim 3, further comprising said pocket being one of a plurality of said pockets, each of said plurality of pockets being attached to said outer surface of said pocket panel.

6. The device of claim 1, further comprising said strap having a first end and a second end, each of said first end and said second end of said strap being attached to said main panel proximate said second end of said main panel.

7. The device of claim 2, further comprising said pocket panel being positioned on said main panel proximate said first end of said main panel, said pocket panel extending between a first longitudinal side and a second longitudinal side of said main panel.

8. The device of claim 1, further comprising:
said sleeve defining a first section including said first end of said main panel and a second section including said second end of said main panel; and
said first section being attached to said second section proximate said first end and said second end of said main panel.

9. The device of claim 8, further comprising an interior surface of said first section abutting an interior surface of said second section between a top of an opened-portion of said channel and each of said first end and said second end of said main panel.

10. The device of claim 1, further comprising an adjustment member coupled to said strap for selectively adjusting a length of said strap.

11. The device of claim 5, further comprising:
each of said pockets having a pair of sides coupled to and extending between said open top end and said closed bottom end of said associated pocket; and
said sides of a pair of said pockets being elongated and extending vertically between a top and a bottom of said pocket panel.

12. The device of claim 11, further comprising a third one of said pockets being positioned between said pair of said pockets, said sides of said third one of said pockets extending vertically between said top and said bottom of said pocket panel.

13. The device of claim 2, further comprising a reinforcement panel being coupled to said exterior surface of said main panel proximate said second end of aid main panel and being positioned opposite said pocket panel, said reinforcement panel being of increased strength relative to said pocket panel and said main panel.

14. The device of claim 5, further comprising said closure extending along a length of said pocket panel, said closure being positioned above said open top end of said pockets.

15. The device of claim 1, further comprising a pillow being postionable within said channel.

16. The device of claim 15, further comprising:
a first end of said pillow extending outwardly beyond a first longitudinal side of said main panel when said pillow is centrally positioned within said channel; and
a second end of said pillow extending outwardly beyond a second longitudinal side of said main panel when said pillow is centrally positioned within said channel.

17. The device of claim 15, further comprising said pillow being elongated and having a main section, a first end and a second end, said main section being annular, each of said first end and said second end of said pillow being convexly arcuate.

18. The device of claim 9, further comprising a rod being positioned between said first section and said second section of said main panel in a void above said channel, said rod abutting said interior surfaces of each of said first section and said second section of said main panel.

19. The device of claim 1, further comprising:
said strap having a break therein defining first and second strap portions; and
a coupler removably coupling said first strap portion to said second strap portion for selectively retaining said strap in a closed loop orientation.

20. A neck-worn arm rest device comprising:
a main panel having a first end and a second end, said main panel being positioned to form a sleeve, said sleeve defining a channel configured for receiving both arms of a user therein to support the arms within said channel, said sleeve defining a first section including said first end of said main panel and a second section including said second end of said main panel, said first section being attached to said second section proximate said first end and said second end of said main panel, an interior surface of said first section abutting an interior surface of said second section between a top of an opened-portion of said channel and each of said first end and said second end of said main panel, said main panel being flexible;
a strap coupled and extending outwardly from said main panel, said strap being configured for being worn around a neck of the user, said strap having a first end and a second end, each of said first end and said second end of said strap being attached to said main panel proximate said second end of said main panel, said strap having a break therein defining a first strap portion including said first end of said strap and a second strap portion including said second end of said strap;
a coupler removably coupling said first strap portion to said second strap portion for selectively retaining said strap in a closed loop orientation, said coupler comprising a buckle;
an adjustment member coupled to said strap for selectively adjusting a length of said strap;
a pocket being coupled to an exterior surface of said main panel, said pocket having an open top end a closed bottom end, said pocket being one of a plurality of said pockets, each of said pockets having a pair of sides coupled to and extending between said open top end and said closed bottom end of said associated pocket;
a pocket panel being coupled to said exterior surface of said main panel, each of said plurality of pockets being attached to said outer surface of said pocket panel, said pocket panel having an opening therein for accessing an interior of said pocket panel, said pocket panel being positioned on said first section of said main panel proximate said first end of said main panel, said pocket panel extending between a first longitudinal side and a second longitudinal side of said main panel;
a reinforcement panel being coupled to said exterior surface of said main panel proximate said second end of said main panel and being positioned opposite said pocket panel, said reinforcement panel being of increased strength relative to said pocket panel and said main panel;
wherein said sides of a pair of said pockets are elongated and extend vertically between a top and a bottom of said pocket panel;
a third one of said pockets being positioned between said pair of said pockets, said sides of said third one of said pockets extending vertically between said top and said bottom of said pocket panel;
a closure attached to said pocket panel and being actuated to a closed state closing said opening or to an opened state allowing access to said interior of said pocket panel, said closure comprising a zipper, said closure extending along a length of said pocket panel, said closure being positioned above said open top end of said pockets; and
a pillow being postionable within said channel, said pillow being elongated and having a main section, a first end and a second end, said main section being annular, each of said first end and said second end of said pillow being convexly arcuate, said first end of said pillow extending outwardly beyond said first longitudinal side of said main panel when said pillow is centrally positioned within said channel, said second end of said pillow extending outwardly beyond said second longitudinal side of said main panel when said pillow is centrally positioned within said channel.
